(19) United States
(12) Patent Application Publication
(10) Pub. No.: US 2026/0053350 A1
(43) Pub. Date: Feb. 26, 2026

(54) OPHTHALMIC APPARATUS

(71) Applicant: NIDEK CO.,LTD., Aichi (JP)

(72) Inventor: Seiji MASEGI, Aichi (JP)

(73) Assignee: NIDEK CO., LTD., Aichi (JP)

(21) Appl. No.: 19/298,775

(22) Filed: Aug. 13, 2025

(30) Foreign Application Priority Data
Aug. 26, 2024 (JP) 2024-143995

(51) Int. Cl. A61B 3/107; A61B 3/00

(52) U.S. Cl. CPC A61B 3/107; A61B 3/0008

(57) ABSTRACT

An ophthalmic apparatus has an examination window, a first optical system which projects a first pattern index with visible light onto a subject eye from an outside of the examination window, a second optical system which projects a second pattern index with infrared light onto the subject eye from an inside of the examination window, an imaging optical system including an imaging element which receives a first cornea reflection image caused by the first pattern index and a second cornea reflection image caused by the second pattern index via an objective lens, and a coupler which couples optical paths of the imaging optical system and the second optical system. The coupler is an optical element having a characteristic in which an attenuation ratio of the visible light is smaller than that of the infrared light in an optical path from a cornea of the subject eye toward the imaging element.

### CROSS-REFERENCE TO RELATED APPLICATIONS

This application is based on and claims priority under 35 USC 119 from Japanese Patent Application No. 2024-143995 filed on August 26, 2024, the entire content of which is incorporated herein by reference.

### TECHNICAL FIELD

The present disclosure relates to an ophthalmic apparatus that measures a corneal shape of a subject eye.

### BACKGROUND ART

In the related art, an ophthalmic apparatus for measuring a corneal shape of a subject eye is known (see JPH08-280624A). In the ophthalmic apparatus for measuring the corneal shape as disclosed in JPH08-280624A, an objective lens and an examination window may be enlarged in order to expand a measurement range so as to add the function of an anterior segment observation system. When the objective lens and the examination window are enlarged, a diameter of an innermost side of a pattern index projected from the outside of the examination window increases, and a measurement accuracy of the corneal shape of the central portion of the subject eye may decrease. Therefore, in order to maintain the measurement accuracy, in addition to projecting the pattern index from the outside of the examination window, the pattern index may be projected from also the inside of the examination window to the central portion of the cornea.

However, when the pattern index optical system is arranged inside the examination window, the amount of light of the pattern index projected from the outside of the examination window of the ophthalmic apparatus, which reaches the imaging optical system, may be attenuated due to an influence of an optical path coupling unit used in the ophthalmic apparatus.

### SUMMARY OF INVENTION

A typical object of the present disclosure is to provide an ophthalmic apparatus capable of sufficiently securing the amount of light of a pattern index that reaches an imaging optical system and is projected onto a subject eye from the outside of an examination window.

According to an aspect of the present disclosure, there is provided an ophthalmic apparatus having: an examination window; a first pattern index projection optical system configured to project a first pattern index onto a subject eye from an outside of the examination window; a second pattern index projection optical system configured to project a second pattern index onto the subject eye from an inside of the examination window; an imaging optical system including an imaging element configured to receive a first cornea reflection image caused by the first pattern index and a second cornea reflection image caused by the second pattern index via an objective lens; and an optical paths coupler configured to couple optical paths of the imaging optical system and the second pattern index projection optical system, in which the first index projection optical system projects the first pattern index with visible light, the second index projection optical system projects the second pattern index with infrared light, and the optical paths coupler is an optical element having a characteristic in which a ratio of attenuation of the visible light is smaller than a ratio of attenuation of the infrared light in an optical path from a cornea of the subject eye toward the imaging element.

### BRIEF DESCRIPTION OF DRAWINGS

Exemplary embodiment(s) of the present invention will be described in detail based on the following figures, wherein:

FIG. 1 is a diagram illustrating a configuration of an ophthalmic apparatus;

FIG. 2 is a diagram schematically illustrating an optical system of the ophthalmic apparatus and a control system of the ophthalmic apparatus;

FIG. 3 is a diagram for explaining an pattern index projection unit from the front direction on the side where an examinee is positioned;

FIG. 4 is a cross-sectional view of a surface emitting panel;

FIG. 5 is a flowchart illustrating steps of corneal shape measurement;

FIG. 6 is a graph showing luminance information of an image obtained by converting a color image into a grayscale image;

FIGS. 7A to 7C are graphs showing luminance information of each color channel image;

FIG. 8 is an image of a cornea of a subject eye on which a pattern index is projected; and

FIG. 9 is an image of a shape map based on corneal shape information.

### DESCRIPTION OF EMBODIMENTS

An ophthalmic apparatus (for example, ophthalmic apparatus 1) of the present disclosure includes an examination window, an illumination optical system, an imaging optical system, an optical paths coupler, a color channel separation unit, a controller, and the like. An examination light of an anterior segment observation system, a reflected light from the anterior segment, and the like pass through the examination window (for example, examination window 18). The illumination optical system (for example, first pattern index projection optical system 20 and second pattern index projection optical system 70) projects a pattern index onto a cornea of a subject eye. The optical paths coupler (for example, dichroic mirror 74) guides a light flux of the illumination optical system inside the examination window to the cornea of the subject eye and an imaging element. The imaging optical system (for example, front imaging optical system 60) is used to capture an index image of a pattern index projected onto the cornea of the subject eye. The imaging optical system includes the imaging element. The color channel separation unit (for example, controller 2) separates an image of the captured index image of the pattern index into respective color channels such as a red channel, a green channel, and a blue channel. The controller (for example, controller 2) is used to weight an image of the index image of the pattern index captured by the imaging optical system, and acquire corneal shape information of the subject eye.

The ophthalmic apparatus of the present embodiment includes an examination window, a first pattern index projection optical system configured to project a first pattern index onto a subject eye from the outside of the examination window, and a second pattern index projection optical system configured to project a second pattern index different from the first pattern index onto the subject eye from the inside of the examination window. The first cornea reflection image caused by the first pattern index projected from the first pattern index projection optical system and the second cornea reflection image caused by the second pattern index projected from the second pattern index projection optical system are captured by an imaging optical system including an imaging element via an objective lens. The first pattern index projected from the outside of the examination window is projected with visible light, and the second pattern index projected from the inside of the examination window is projected with infrared light. As a result, an optical element having a characteristic in which a ratio of attenuation of the visible light is smaller than a ratio of attenuation of the infrared light in an optical path from a cornea of the subject eye to the imaging element can be adopted as an optical paths coupler that couples optical paths of the imaging optical system and the second pattern index projection optical system. As a result, the first corneal reflection image caused by the first pattern index can be efficiently captured. Furthermore, a light source of the first pattern index projection optical system can be easily simplified.

The outside of the examination window means the outside including an exterior portion of a housing of the ophthalmic apparatus 1. The inside of the examination window means the inside of the housing of the ophthalmic apparatus 1. Further, the characteristic in which the attenuation ratio of the optical element is small indicates a characteristic in which the loss of a light amount due to an influence of reflectance and transmittance of the optical element with respect to predetermined light is small. That is, the optical element is an optical element having a characteristic in which light reception efficiency of the imaging element for the visible light is higher than that for the infrared light. The optical path from the cornea of the subject eye to the imaging element indicates an optical path from the objective lens to the imaging element.

For example, in the ophthalmic apparatus, the second pattern index is a ring-shaped pattern. Accordingly, a corneal shape of a central portion of the cornea of the subject eye can be measured using the second pattern index.

Further, for example, in the ophthalmic apparatus, a wavelength of a light source of the second pattern index projection optical system is identical to a wavelength of a light source of the anterior segment illumination optical system. The term “identical” as used herein is not necessarily limited to being completely identical. The term “identical” as used herein includes being substantially the same to the extent that no significant difference occurs. Thus, the wavelength of the light source of the second pattern index projection optical system does not overlap that of the other measurement light source mounted on the ophthalmic apparatus. When the second pattern index is projected at a wavelength used for another measurement light source, optical path coupling or the like in the optical paths coupler inside the ophthalmic apparatus may be affected. However, the optical paths coupler can be easily designed by adopting a configuration in which the second pattern index is projected at a wavelength different from those of the other measurement light sources.

Further, for example, the ophthalmic apparatus turns off the light source of the anterior segment illumination optical system while the second pattern index projection optical system projects the second pattern index. This prevents unevenness from occurring in the pattern index due to anterior segment illumination when the pattern index is projected onto the subject eye.

Further, for example, the controller may acquire corneal shape information of the subject eye by performing weighting processing for each subject eye on the image separated by the separation unit. This makes it possible to acquire the corneal shape information using an image subjected to image processing in accordance with features of the subject eye. Therefore, the influence of iris color of the subject eye on the acquisition of the corneal shape information can be reduced.

Further, for example, the controller may acquire the corneal shape information of the subject eye by separating an image obtained by capturing, in the imaging optical system, an index image of a pattern index projected onto the cornea of the subject eye by the illumination optical system into a plurality of color channels, and by performing weighting processing based on luminance information of each color channel image. Accordingly, the contrast between the iris and the index image of the pattern index can be ensured regardless of the iris color, and the measurement accuracy of the corneal shape of the subject eye can be improved. For example, when an image obtained by projecting a pattern index with white light is captured, the image is separated into color channels of red, green, and blue. By multiplying a color channel having a high contrast between the iris and the index image of the pattern index by a large weighting coefficient based on the luminance information of each color channel, an image having a high contrast between the iris and the index image of the pattern index can be synthesized. In addition, since the corneal shape information of the subject eye is acquired by performing image processing after imaging, it is possible to perform good corneal shape measurement with one imaging.

Further, for example, the controller may acquire the corneal shape information of the subject eye by performing weighting processing on each color channel image based on the contrast of each color channel image separated into the plurality of color channels by the separation unit. Among the color channel images, a color channel image having a high contrast has a large weight value, and a color channel image having a low contrast has a small weight value. That is, the image processing is performed with emphasis on a color channel image having high contrast. Accordingly, the contrast of the image generated by the image processing is also increased, and the measurement accuracy of the corneal shape of the subject eye can be improved.

In addition, for example, the controller may acquire iris color information of the subject eye and perform weighting processing on the color channel image based on the iris color information to acquire the corneal shape information of the subject eye. For example, image processing can be performed using a weighting coefficient set in advance in accordance with the iris color of the subject eye. Accordingly, since the color channel image can be processed using the iris color information, more accurate image processing can be performed.

The corneal shape information may include, for example, an anterior segment image of the subject eye onto which an index image of a pattern index is projected. The anterior segment image of the subject eye is obtained by performing weighting processing on a color channel image obtained by separating an image captured by the imaging optical system for each color channel. The image acquired by the ophthalmic apparatus may be the weighted color channel image itself. Alternatively, a color channel image subjected to weighting processing may be combined by image processing. In addition, the corneal shape information may include, for example, a parameter related to the corneal shape.

The ophthalmic apparatus performs weighting processing on the plurality of color channel images obtained by separating the captured image, and outputs the acquired corneal shape information when the corneal shape information is acquired. As an output method, for example, corneal shape information may be displayed on a display. It could be an output method of displaying parameters of corneal shape information in a color map format on an anterior segment image.

Hereinafter, the ophthalmic apparatus according to the present embodiment will be described with reference to the drawings. The drawings to be referred to are used to describe technical features that can be adopted by the present disclosure, and configurations and the like to be described are not intended to be limited thereto and are merely explanatory examples.

The ophthalmic apparatus of the present embodiment is an apparatus capable of acquiring corneal shape information of a subject eye. For example, the ophthalmic apparatus may include an optical system used for measuring a corneal shape, a corneal shape information acquisition unit, and the like. Further, for example, the ophthalmic apparatus may include a wavefront aberration information acquisition unit, an axial length information acquisition unit, an anterior segment information acquisition unit, an eye refractive power information acquisition unit, and the like.

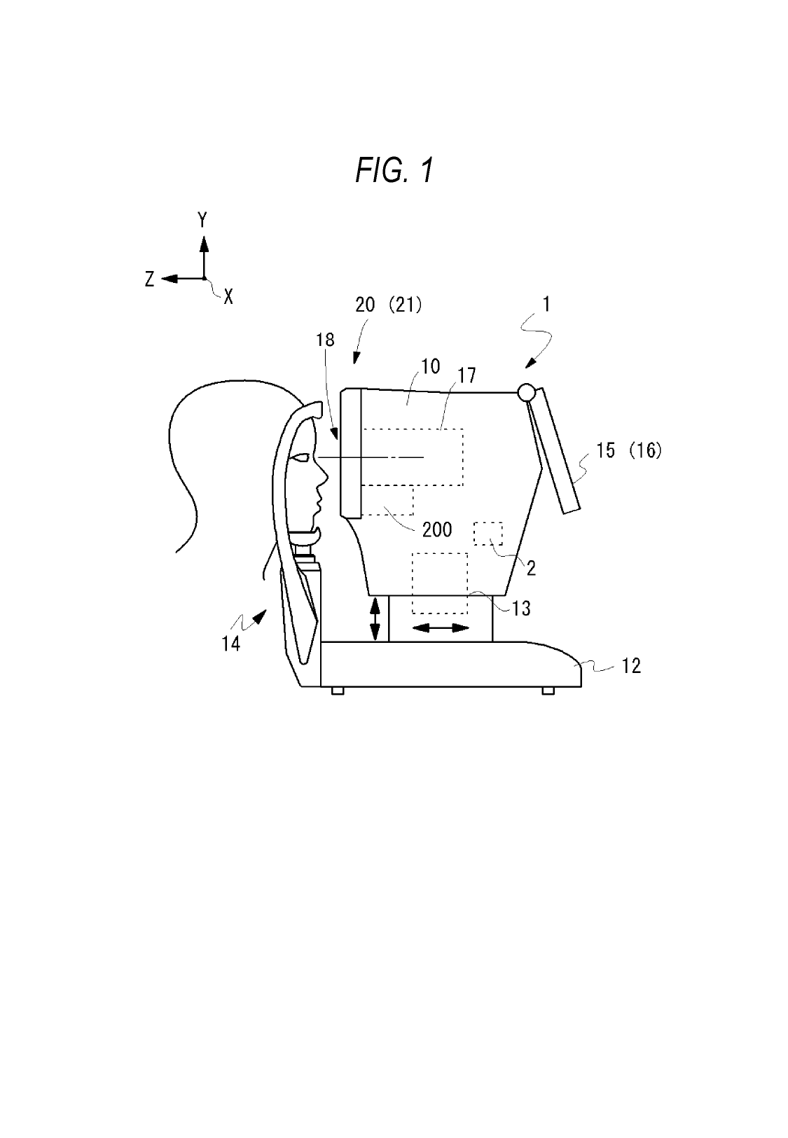

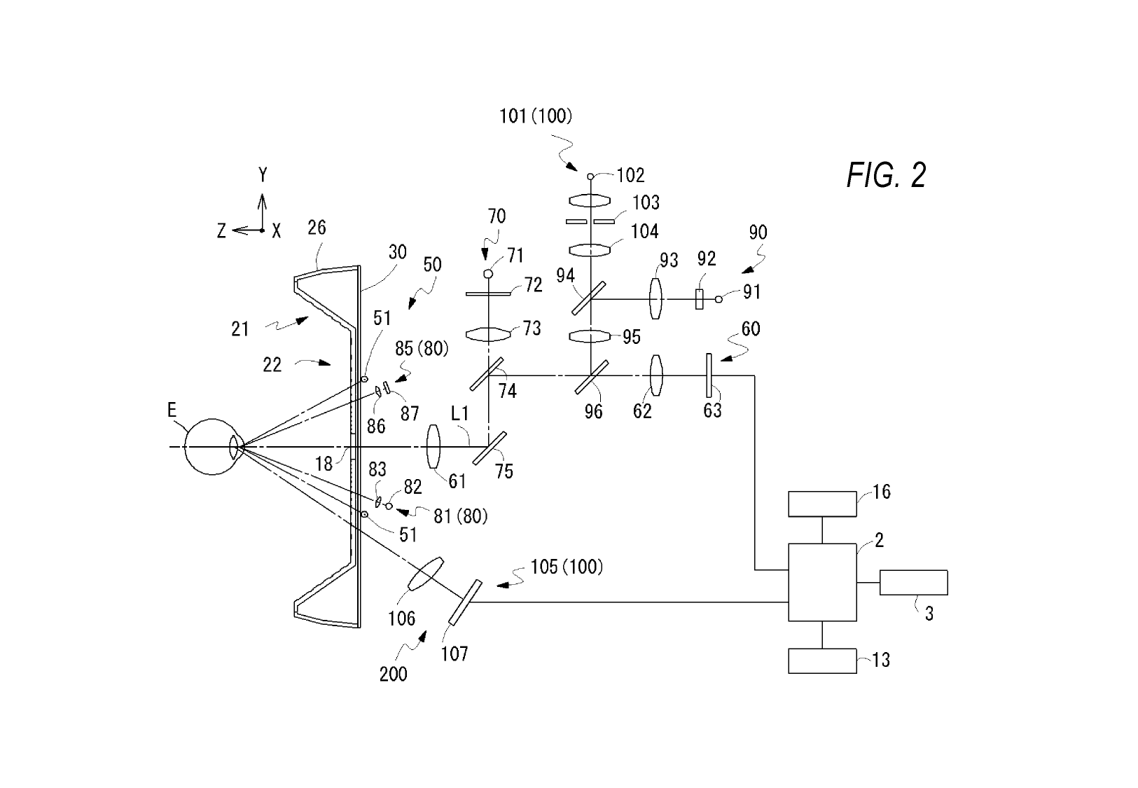

FIG. 1 is an external view of an ophthalmic apparatus 1. The ophthalmic apparatus 1 includes a controller 2, an optometry unit 10, a base 12, a driver 13, a face support unit 14, a display 15, an operation unit 16, a corneal shape measurement unit 17, and an examination window 18. The optometry unit 10 includes the corneal shape measurement unit 17 for measuring a corneal shape of a subject eye E. A first pattern index projection optical system 20 of the corneal shape measurement unit 17 is disposed on the front side (subject eye side) of the optometry unit 10. The first pattern index projection optical system 20 is disposed to face the subject eye. The corneal shape measurement unit 17 is provided with the examination window 18 centered on a measurement optical axis L1 (see FIG. 2). In addition to the corneal shape measurement unit 17, the ophthalmic apparatus 1 may include an examination unit for examining (measuring or imaging) optical characteristics of the subject eye other than the corneal shape measurement. In the present example, an example in which the optometry unit 10 includes an anterior segment cross-sectional imaging unit 200 (see FIG. 2) will be described.

The driver 13 changes a positional relationship of the optometry unit 10 with respect to the subject eye. For example, the driver 13 moves the optometry unit 10 in an X direction (left-right direction), a Y direction (up-down direction), and a Z direction (front-rear direction) with respect to the subject eye E by three-dimensionally moving the optometry unit 10 with respect to the base 12. The face support unit 14 is used to fix an examinee's face in front of the optometry unit 10. The face support unit 14 is fixed to the base 12, and fixes the subject eye E by supporting the face of the examinee.

FIG. 2 is a schematic configuration diagram of an optical system disposed in the optometry unit 10 and a control system of the ophthalmic apparatus 1. The corneal shape measurement unit 17 includes a first pattern index projection optical system 20 for measuring the corneal shape of the subject eye E, a front imaging optical system 60, a working distance detection optical system 80, a second pattern index projection optical system 70, and a fixation index presentation optical system 90. The anterior segment cross-section imaging unit 200 includes a cross-section imaging optical system 100.

The first pattern index projection optical system 20 includes a first pattern index projection unit 21. The first pattern index projection unit 21 includes a first pattern index plate 22 and a surface-emitting panel 30. Further, the first pattern index projection unit 21 may additionally include a reflection plate 26 on an outer peripheral portion of the first pattern index plate 22.

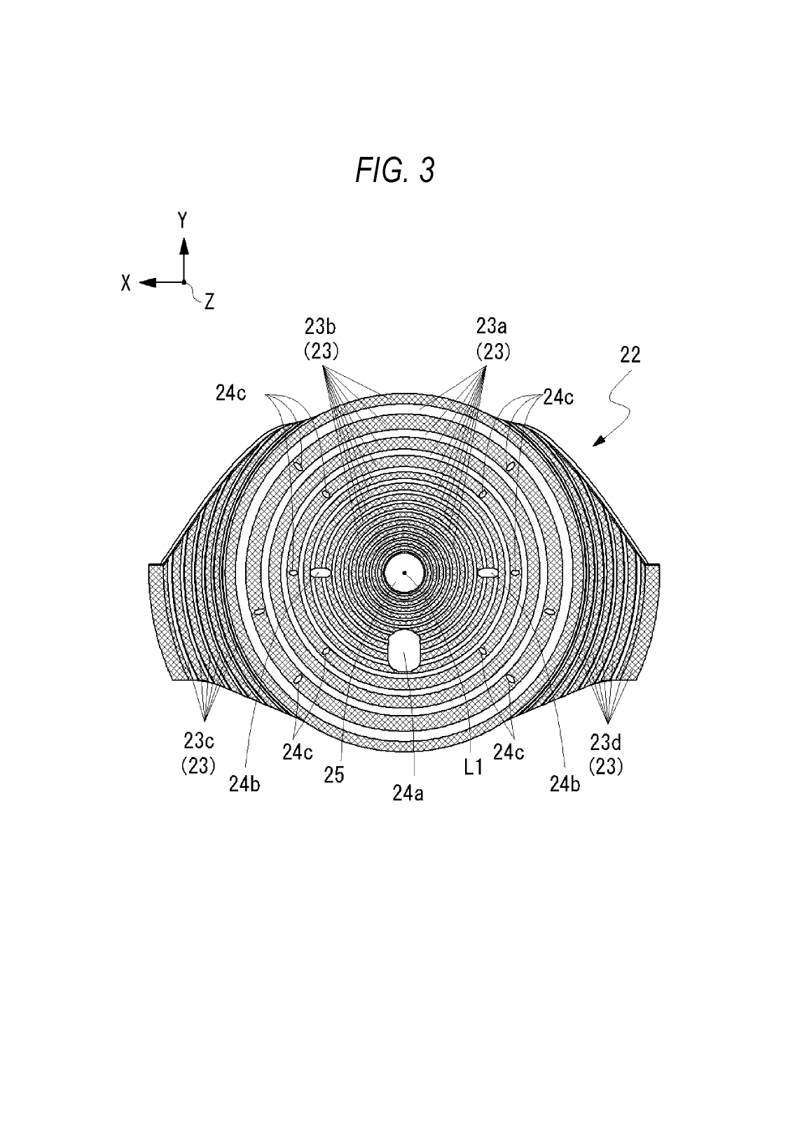

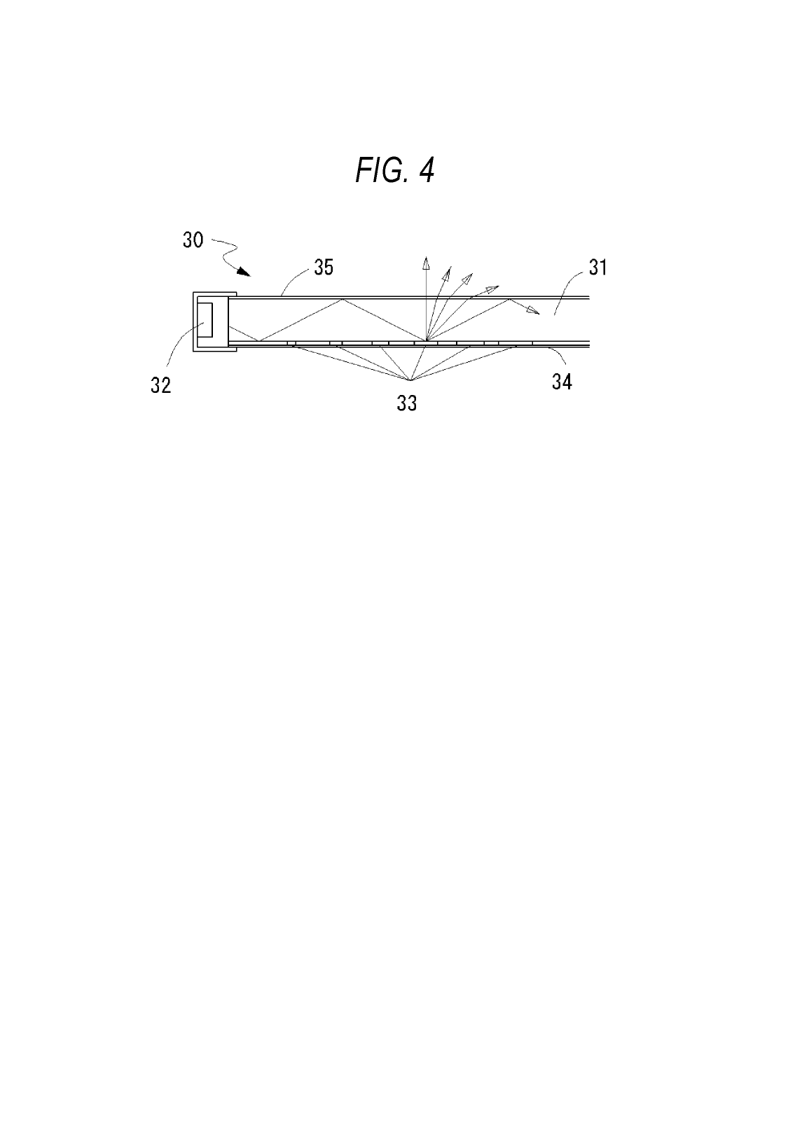

FIG. 3 is a diagram illustrating a configuration of the first pattern index plate 22. FIG. 3 illustrates the first pattern index projection unit 21 from the front direction on the side where the examinee is positioned. The surface-emitting panel 30 (see FIG. 4) is disposed behind the first pattern index plate 22. The first pattern index plate 22 is formed of a plate of a translucent body (for example, acrylic resin) that guides illumination light from the surface emitting panel 30. In the first pattern index plate 22, a first pattern index 23 for obtaining a corneal shape is formed on a plate of a translucent body. In the present example, the first pattern index 23 is a multiple ring-shaped pattern index as shown in FIG. 2. In the first pattern index 23, a plurality of ring-shaped light shielding portions 23b coated in black and a plurality of ring-shaped light transmitting portions 23a not coated are alternately formed concentrically around the measurement optical axis L1 on the first pattern index plate 22. The pattern plate 120 includes a light transmitting portion 23a and a light shielding portion 23b having a flat plate shape, and a light transmitting portion 23c and a light shielding portion 23d which are arranged on the outer peripheral side of the light transmitting portion 23a and the light shielding portion 23b. The light transmitting portion 23c is not coated, and the light shielding portion 23d is coated with black. The transparent portion 23a and the light shielding portion 23b include a central opening 25 through which the measurement optical axis L1 of the front imaging optical system 60 passes, and have a circular shape around the center of the central opening 25, and the ring-shaped first pattern index 23 is formed on substantially the entire surface.

FIG. 4 is a diagram illustrating a configuration of the surface emitting panel 30. The surface emitting panel 30 includes a light guide plate 31, an illumination light source 32, a reflection pattern 33, a reflection sheet 34, and a diffusion plate 35 as main elements.

The light guide plate 31 is formed of a translucent body (for example, acrylic resin). The light from the surface emitting panel 30 passes through the light transmitting portion 23a and the light transmitting portion 23c, and projects the Placido ring pattern of the first pattern index 23 onto a cornea Ec of the subject eye. In the surface emitting panel 30, in addition to a central opening (not shown) through which a light flux for the corneal shape measurement passes, various light passing holes (not shown) through which a light flux of the cross-sectional imaging optical system 210, a light flux of the working distance detection optical system 80, and illumination light for illuminating the subject eye at the time of alignment and observation of the subject eye pass are formed similarly to the first pattern index plate 22. The optical system that illuminates the light transmitting portion 23a and the light transmitting portion 23c of the first pattern index 23 from behind may be, for example, an optical system in which a ring-shaped light source is provided behind the light transmitting portion 23a and the light transmitting portion 23c.

As the illumination light source 32, for example, an LED that emits white light including light of various wavelengths of 400 nm to 700 nm is used. The illumination light source 32 illuminates the first pattern index plate 22. A plurality of illumination light sources 32 are arranged at the end of the light guide plate 31. For example, the reflection pattern 33 may be formed in a shape of reflection dots in which paint having reflection properties is printed in a dot shape on the back surface side of the light guide plate 31.

The reflection sheet 34 is disposed on the back surface of the surface light-emitting panel 30 with the reflection pattern 33 interposed therebetween. Accordingly, the light from the illumination light source 32 guided by the light guide plate 31 is reflected to the front surface side. The diffusion plate 135 is disposed on the front surface side of the surface light-emitting panel 30 and diffuses light emitted from the front surface of the surface light-emitting panel 30.

Returning to FIG. 2, an anterior segment illumination optical system 50 includes an illumination light source 51 that emits infrared light or white light. In the example, the illumination light source 51 is embedded in the first pattern index projection unit 21. In the first pattern index plate 22, six light passing holes 24c are formed around the measurement optical axis L1 in the 0-degree direction, the 45-degree direction, the 135-degree direction, the 180-degree direction, the 225-degree direction, and the 315-degree direction on the first concentric circle outside the light passing hole 24b, and four light passing holes 24c are formed in the 45-degree direction, the 135-degree direction, the 225-degree direction, and the 315-degree direction on the second concentric circle outside the first concentric circle. Since the illumination light source 51 illuminates the subject eye E through the light passing hole 24c, the illumination light source 51 is also provided at the same position as the light passing hole 24c in the first pattern index plate 22.

The front imaging optical system 60 includes an objective lens 61, an imaging lens 62, and an imaging element 63 on the measurement optical axis L1. The front imaging optical system 60 is a device that captures an image. The front imaging optical system 60 is, for example, a color camera. The front imaging optical system 60 is disposed, for example, at a pupil conjugate position of the anterior segment of the subject eye. The front imaging optical system 60 is used to capture an index image of the first pattern index 23 projected onto the cornea of the subject eye when measuring the corneal shape. A front image of the anterior segment of the subject eye illuminated by the anterior segment illumination light source 50 is captured by the imaging element 63. The front imaging optical system 60 also serves as a detection optical system that detects the index image projected onto the cornea Ec of the subject eye by the second pattern index projection optical system 70. The index image formed by the second pattern index projection optical system 70 is captured by the imaging element 63.

The imaging element 63 is an imaging element capable of simultaneously acquiring light of each wavelength. The imaging element 63 is disposed such that the imaging surface is located on the image plane of the imaging lens 62 in the front imaging optical system 60. The imaging element 63 may be, for example, a charge-coupled device (CCD) image sensor. The imaging element 63 may include a microlens, a color filter, a photodiode, and the like (all not illustrated in the drawings). The microlens focuses light on the photodiode. The color filter may have a Bayer array in which a column in which the red filter and the green filter are arranged and a column in which the green filter and the blue filter are arranged are alternately arranged. In addition, a color filter using an arrangement method other than the Bayer arrangement may be used. The photodiode converts the received light into an electric signal. The imaging element 63 captures an image formed on the imaging surface via the imaging lens 62 and generates an image signal indicating the captured image.

In the front imaging optical system 60, the imaging light flux from the light source 71 passes through the second pattern index plate 72 and the lens 73, passes through the dichroic mirror 74 which is an optical element, is coaxial with the measurement optical axis L1, is reflected by a mirror 75, and reaches the cornea Ec of the subject eye via the objective lens 61 and the examination window 18. The return light from the cornea Ec reaches the imaging element 63 via the objective lens 61, the mirror 75, the dichroic mirror 74, a half mirror 96, and the imaging lens 62. Thereby, an index image of the pattern index projected on the cornea Ec of the subject eye is acquired.

The dichroic mirror 74 is an optical element that transmits a part of the light flux of the light source 71 between the subject eye E and the imaging element 63 to transmit the first cornea reflection image and the second cornea reflection image. The dichroic mirror 74 has a split ratio of reflection and transmission in which a ratio of attenuation of the first pattern index projected with visible light is small with respect to a ratio of attenuation of the second pattern index projected with infrared light in the optical path from the cornea Ec of the subject eye to the imaging element 63.

The dichroic mirror 74 may have, for example, a characteristic of having a transmittance of about 80% for infrared light having a wavelength of 700 nm to 900 nm. Further, it may have a characteristic that the transmittance for light having a wavelength shorter than 600 nm and light having a wavelength longer than 920 nm is about 20%. In this case, in the dichroic mirror 74, infrared light for projecting the second pattern index and visible light for projecting the first pattern index have an attenuation ratio of A:B (where A>B).

The working distance detection optical system 80 includes a light projection optical system 81 and a light reception optical system 85. The light projection optical system 81 includes a light source 82 that emits infrared light and a lens 83. The light reception optical system 85 includes a lens 86 and a light reception element 87. The working distance detection optical system 80 is used to align the optometry unit 10 in a working distance direction (Z direction) with respect to the subject eye.

The light from the light source 82 is converted into a substantially parallel light flux by the lens 83, passes through a light passage hole (not shown) formed in the surface emitting panel 30 and the light passage hole 24b formed in the first pattern index plate 22, and is irradiated to the cornea of the subject eye from an oblique direction. The optical axis of the light reception optical system 85 is provided so as to be symmetrical to the optical axis of the light projection optical system 81 with respect to the measurement optical axis L1. The corneal reflection light from the light projection optical system 81 is incident on the light reception element 87 via the light passing hole 24b formed in the first pattern index plate 22, a light passing hole (not shown) formed in the surface emitting panel 30, and the lens 86. When the subject eye relatively moves in the working distance direction (Z direction), the index image (image of the light source 82) formed on the cornea Ec of the subject eye also moves on the light reception element87. Therefore, the alignment state of the optometry unit 10 with respect to the subject eye in the working distance direction is detected by detecting the index image on the light reception element 87.

The second pattern index projection optical system 70 includes a light source 71 that emits infrared light, a second pattern index plate 72, a lens 73, and a dichroic mirror 74. For example, a double ring index is formed on the second pattern index plate 72. The light flux of the ring index on the second pattern index plate 72 illuminated by the light source 71 is made coaxial with the measurement optical axis L1 via the lens 73 and the dichroic mirror 74, and is projected onto the cornea of the subject eye. As a result, an index image of the second pattern index is formed on the cornea Ec. The index image of the second pattern index projected on the cornea Ec of the subject eye is received by the imaging element 63 of the front imaging optical system 60 as a second cornea reflection image. The second corneal reflection image may be received by the imaging element 63 and subjected to image processing by the controller 2 to be used for detecting the alignment state in the XY directions of the relative positional relationship between the subject eye and the optometry unit 10.

The fixation index presentation optical system 90 presents a fixation index to the subject eye. The fixation index presentation optical system 90 includes at least a light source 91 that emits visible light and a fixation index plate 92. The fixation light flux from the light source 91 passes through the fixation index plate 92, a lens 93, a half mirror 94, and a lens 95, and is reflected by a half mirror 96 to be coaxial with the measurement optical axis L1. Thereafter, the fixation light flux reaches the fundus of the subject eye through the objective lens 61.

The cross-sectional imaging optical system 100 is used to capture a cross-sectional image of the anterior segment. The cross-sectional imaging optical system 100 includes a light projection optical system 101 and a light reception optical system 105.

The light projection optical system 101 is coaxial with the measurement optical axis L1 and projects slit light, which is an example of imaging light, to the anterior segment. The light projection optical system 101 includes a light source 102 and a slit 103. The light source 102 uses, for example, red visible light or near-infrared light as imaging light. The slit 103 may be arranged at the pupil conjugate position. For example, the slit 103 is disposed so as to optically cut the slit light in the horizontal direction (X direction) of the anterior segment.

The light reception optical system 105 includes a lens system 106, an imaging element 107 as an example of a photodetector, and the like. In the light reception optical system 105, the lens system 106 and the imaging element 107 are arranged in a Scheimpflug relationship with a cutting plane set in the anterior segment. That is, the cutting plane, the principal plane of the lens system 106, and the extended plane of the imaging plane of the imaging element 107 are arranged to intersect at one intersection line (one axis). The imaging element 107 receives return light (reflected light or scattered light) from the anterior segment optically cut by the slit light. Then, a cross-sectional image of the anterior segment is acquired based on the signal from the imaging element 107.

In the cross-sectional imaging optical system 100, the imaging light flux from the light source 102 becomes a slit light flux via the slit 103, and is reflected by the half mirror 96 via the lens 104, the half mirror 94, and the lens 95 to be coaxial with the measurement optical axis L1. Thereafter, the imaging light flux further passes through the dichroic mirror 74, the mirror 75, and the objective lens 61 to reach the anterior segment. The return light from the cut surface formed in the anterior segment reaches the imaging element 107 via the light passage hole 24a formed in the first pattern index plate 22, a light passage hole (not shown) formed in the surface emitting panel 30, and the lens system 106. Thereby, a cross-sectional image of the anterior segment of the subject eye is acquired.

In FIG. 2, the controller 2 controls entirety of the ophthalmic apparatus 1. Various electric elements such as the imaging element 63, the imaging element 107, each light source of the optical system, the display 15, and the driver 13 are connected to the controller 2. The controller 2 also has an image processing function of processing each image captured by the imaging element 63 and the imaging element 107. For example, the controller 2 acquires an image of an index image of a pattern index projected on the cornea Ec of the subject eye at the time of measuring the corneal shape imaged by the imaging element 63, and analyzes the image according to a predetermined program to obtain the corneal shape of the cornea Ec of the subject eye. Further, the controller 2 acquires an anterior segment cross-sectional image captured by the imaging element 107 and analyzes the image in accordance with a predetermined program to obtain the shape of the anterior segment tissue.

The display 15 may function as a touch panel also serving as the operation unit 16. Further, the display 15 displays a measurement result (anterior segment cross-sectional image, measurement result of corneal shape, etc.) of the subject eye on a screen. A storage 3, which is an example of a storage device, is connected to the controller 2. The storage 3 stores the front anterior segment image acquired by the imaging element 63, the cross-sectional image of the anterior segment acquired by the imaging element 107, the measurement result, and the like. Various control programs are stored in the storage 3.

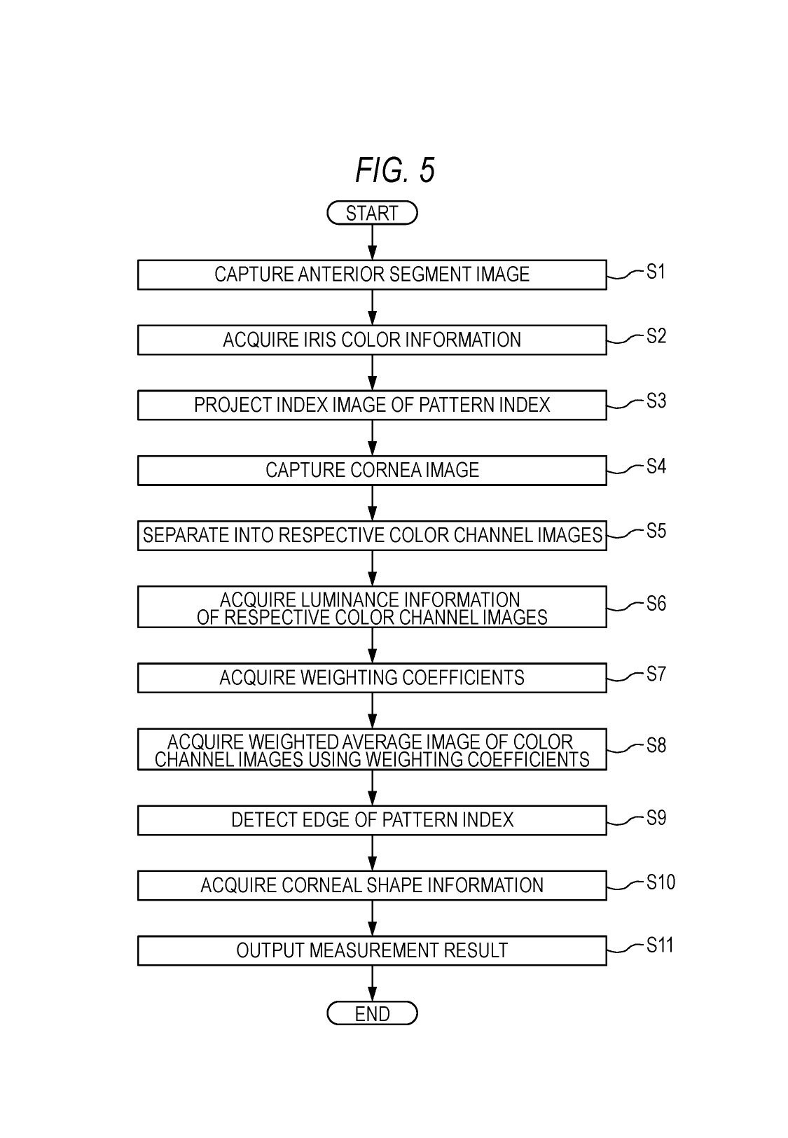

The operation of the ophthalmic apparatus 1 having the above configuration will be briefly described with reference to the flowchart of FIG. 5.

First, the measurement of the corneal shape will be described. When the face of the examinee is supported by the face support unit 14, the front imaging optical system 60 captures a front image of the subject eye E illuminated by the anterior segment illumination optical system 50. The controller 2 controls to display the front image of the subject eye E captured by the front imaging optical system 60 on the display 15. The controller 2 processes the front image of the anterior segment acquired by the front imaging optical system 60, and detects the alignment state of the optometry unit 10 (measurement optical axis L1) with respect to the corneal apex in the X and Y directions based on the index (corneal reflection bright spot) projected onto the cornea Ec of the subject eye by the second pattern index projection optical system 70. The controller 2 detects the alignment state in the Z direction based on the output of the light reception element 87 of the working distance detection optical system 80. Then, the controller 2 controls the driving of the driver 13 to move the optometry unit 10 in the XYZ directions so that the alignment state in the XYZ directions falls within a predetermined allowable range. This completes the alignment of the ophthalmic apparatus 1.

When the alignment is completed, the controller 2 turns on the anterior segment illumination optical system 50 to capture an image of the anterior segment of the subject eye E (S1). The imaging element 63 of the front imaging optical system 60 generates an image signal indicating a captured image and transfers the image signal to the storage 3. The storage 3 stores the image signal generated by the imaging element 63. The controller 2 acquires iris color information of the subject eye by performing image processing for calculating a ratio of color components of the captured image stored in the storage 3 (S2). Here, for example, it is assumed that the controller 2 determines that the iris color of the subject eye is brownish based on the acquired iris color information of the subject eye.

Subsequently, the controller 2 turns on the surface emitting panel 30 and projects the index image of the first pattern index 23 onto the cornea Ec of the subject eye (S3). When the index image is projected onto the cornea Ec of the subject eye, the controller 2 may turn off the anterior segment illumination optical system 50. The controller 2 controls to capture an image of the cornea Ec of the subject eye (S4). The imaging element 63 of the front imaging optical system 60 generates an image signal indicating a captured image and transfers the image signal to the storage 3. The storage 3 stores the image signal generated by the imaging element 63.

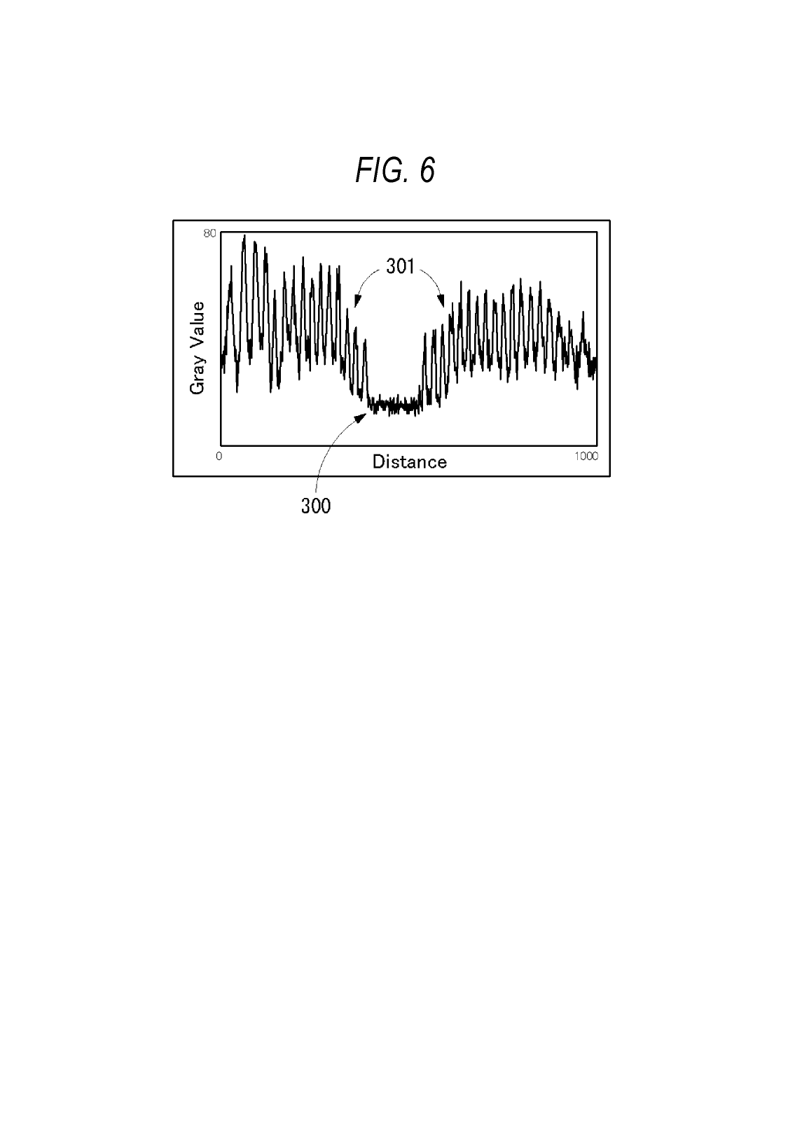

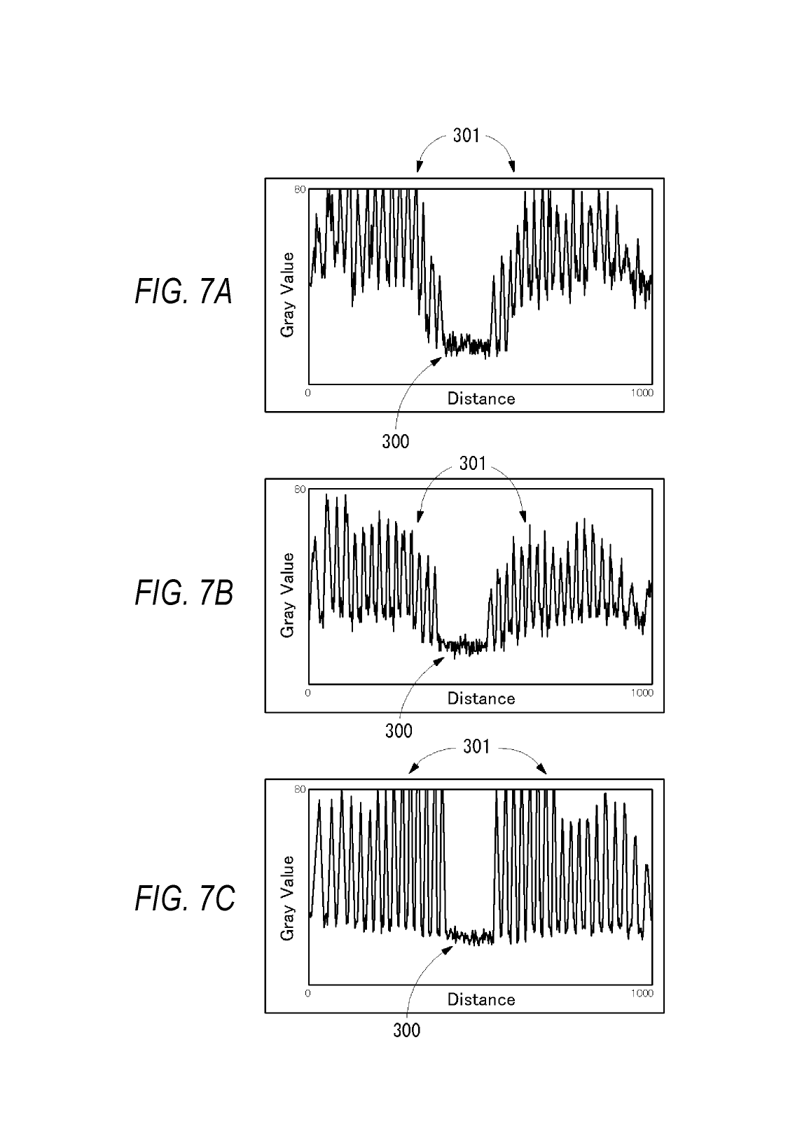

For example, the controller 2 may separate the captured image stored in the storage 3 into three color channel images of a red channel image, a green channel image, and a blue channel image (S5). The controller 2 acquires luminance information for each color channel image in the captured image of the subject eye whose iris color is brownish (S6). FIG. 6 illustrates an example of luminance information of a substantially central portion from the inner corner of the subject eye to the outer corner of the subject eye when the captured image of the subject eye whose iris color is brownish is displayed in gray scale. FIGS. 7A to 7C show examples of luminance information of a substantially central portion from the inner corner to the outer corner of the subject eye in respective color channel images of the captured image (red channel image: FIG. 7A, green channel image: FIG. 7B, blue channel image: FIG. 7C).

In FIGS. 7A to 7C, a range 300 in which the vertical width of the gray value is substantially constant is the pupil range of the subject eye. A range 301 in which the gray values in the luminance information graphs of FIGS. 7A and 7B are on the left and right of the pupil range of the subject eye is the iris range of the subject eye. In FIGS. 7A and 7B, the vertical width of the gray value of the range 301 is indefinite. It is considered that this is because the gray value of the iris range of the subject eye is indefinite in the red channel image and the green channel image. In comparison, the vertical width of the gray value in the range 301 in FIG. 7C is substantially constant. That is, in the blue channel image, the gray value of the iris color of the subject eye is considered to be substantially constant. As described above, when the color image is analyzed for each color channel, the color channel can be separated into a color channel having a large influence of the iris color of the subject eye and a color channel having a small influence of the iris color.

The controller 2 acquires weighting coefficients of respective color channel images set for each iris color based on the acquired iris color information (S7). The weighting coefficients of the red channel image, the green channel image, and the blue channel image acquired by the controller 2 are a weight value a, a weight value b, and a weight value c, respectively. For example, the weighting coefficients for the color channel images set for the subject eye having the brownish iris color may be set to the weight value a = 0, the weight value b = 0.5, and the weight value c = 1.0.

The controller 2 obtains a weighted average image of the three color channel images using the weight value a, the weight value b, and the weight value c (S8). That is, based on the weight values a to c, the controller 2 performs image processing for shifting the color channel image to a color channel image having a larger weight value (that is, shifting the color channel image to a blue channel image). As a result, an image in which the influence of the iris color of the subject eye on the index image of the pattern index is reduced is acquired.

The acquired anterior segment image is stored in the storage 3 or the like. The controller 2 detects a black and white edge of the index image of the pattern index in the processed image stored in the storage 3 (S9), and calculates a corneal curvature for each predetermined angle based on each edge distance from the corneal center. Based on the corneal curvature, a corneal refractive power of the subject eye E is calculated. In this way, the controller 2 acquires, for example, a corneal shape parameter used for outputting a shape map described later (S 10).

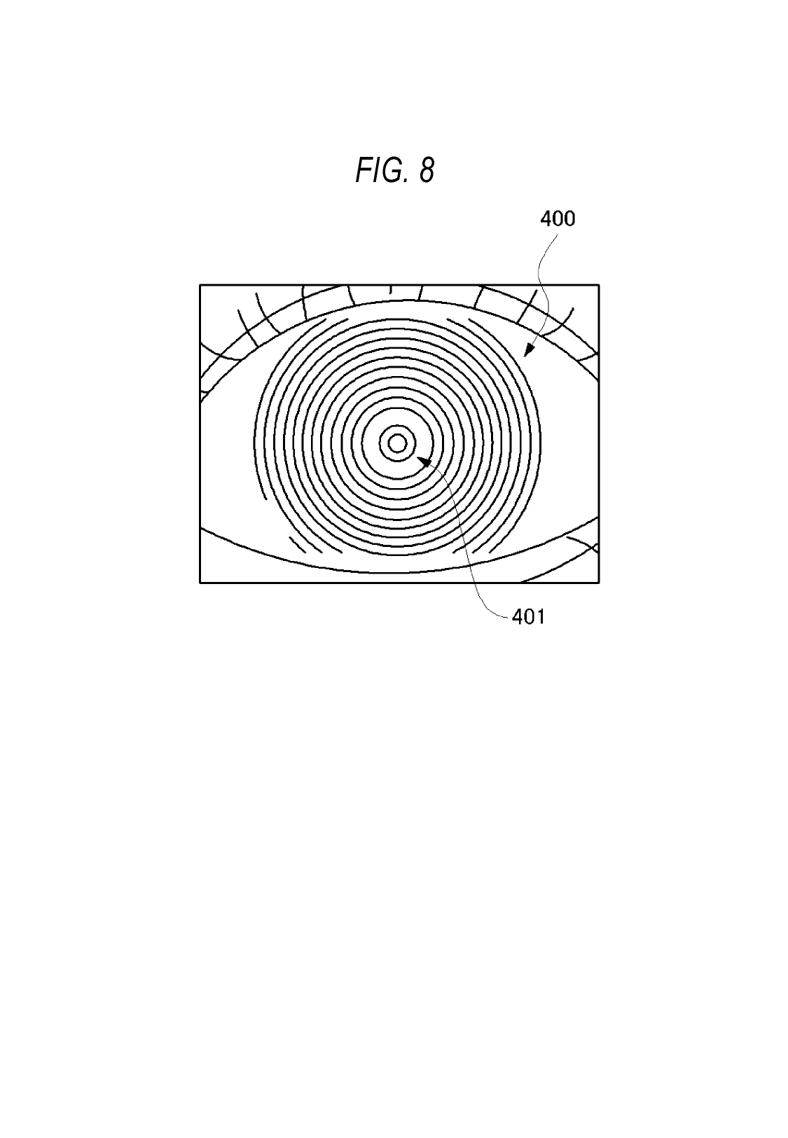

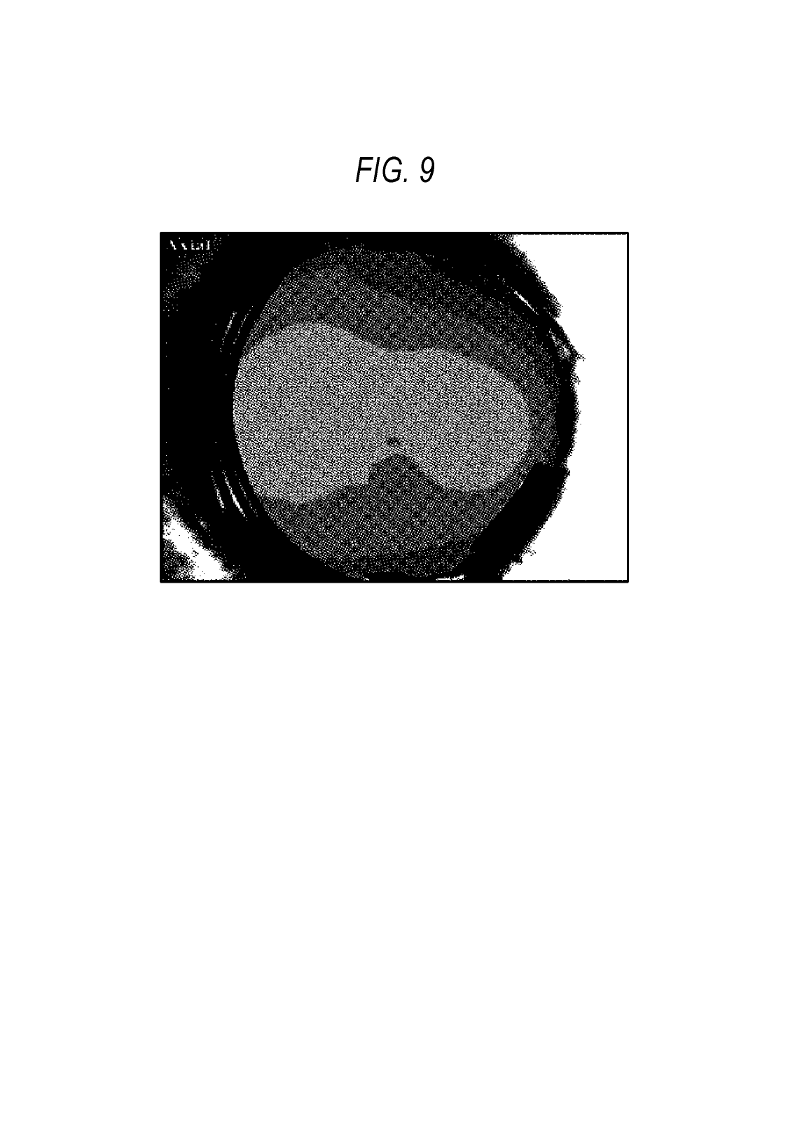

The controller 2 may output the corneal shape information based on the acquired corneal shape parameter of the subject eye E (S 11). For example, the controller 2 may output the measurement result by displaying the measurement result as a shape map on the display 15 (see FIG. 9). The controller 2 may output the corneal shape information by displaying the front image of the subject eye E on which the index image of the pattern index is projected on the display 15 (see FIG. 8).

An operation of measuring the corneal shape of a pupil portion of the subject eye using infrared light may be added to the measurement of the corneal shape. In step S3 of the flowchart of FIG. 5, the controller 2 projects the index image, which is visible light emitted from the surface-emitting panel 30, of the first pattern index onto the cornea Ec of the subject eye using the first pattern index projection optical system 20. At this time, the controller 2 may turn off the anterior segment illumination optical system 50. The first cornea reflection image, which is the return light from the cornea Ec of the subject eye, is received by the imaging element 63 via the objective lens 61, the mirror 75, the dichroic mirror 74, the half mirror 96, and the imaging lens 62.

Further, the controller 2 projects an index image, which is infrared light emitted from the light source 71, of the second pattern index onto the cornea Ec of the subject eye using the second pattern index projection optical system 70. The second cornea reflection image, which is the return light from the cornea Ec of the subject eye, reaches the imaging element 63 via the objective lens 61, the mirror 75, the dichroic mirror 74, the half mirror 96, and the imaging lens 62. More specifically, first, the controller 2 causes the light source 71 to emit light. The light flux of the infrared light from the light source 71 passes through the second pattern index plate 72 and becomes the light flux of the second pattern index. The light flux of the second pattern index passes through the dichroic mirror 74 via the lens 73. The light beam of the second pattern index becomes coaxial with the measurement optical axis L1 by passing through the dichroic mirror 74, and is projected onto the cornea Ec of the subject eye via the mirror 75, the objective lens 61, and the examination window 18. The second cornea reflection image, which is the return light from the cornea Ec of the subject eye, is reflected by the dichroic mirror 74 via the examination window 18, the objective lens 61, and the mirror 75, and is received by the imaging element 63 via the half mirror 96 and the imaging lens 62. The light flux of the second pattern index and the second corneal reflection image are attenuated in light amount when transmitted and reflected by the dichroic mirror 74. In this way, by making the wavelengths of the light of the first pattern index and the second pattern index different from each other, the attenuation of the light amount of the first cornea reflection image attenuated by the dichroic mirror 74 is suppressed, and the first cornea reflection image and the second cornea reflection image can be guided to the imaging element 63.

FIG. 8 is a diagram illustrating an index image of the first pattern index and the second pattern index projected onto the cornea Ec of the subject eye. An index image 400 is an index image of the first pattern index as being the visible light projected onto the cornea Ec of the subject eye when the controller 2 turns on the surface-emitting panel 30. The index image 401 is an index image of the second pattern index as being the infrared light projected onto the cornea Ec of the subject eye when the controller 2 turns on the light source 71 of the second pattern index projection optical system 70. As shown in FIG. 8, by projecting the index image 400 and the index image 401 onto the cornea Ec of the subject eye, the index image can also be projected onto the central portion of the cornea Ec of the subject eye. Therefore, the measurement accuracy of the corneal shape measurement can be maintained as compared with the case where only the first pattern index is projected onto the cornea of the subject eye. That is, the addition of the operation of projecting the index image 400 and the index image 401 onto the cornea Ec leads to the maintenance of the accuracy of the shape measurement of the cornea Ec of the subject eye.

Next, the measurement of the anterior segment cross-sectional shape will be described. The alignment state of the optometry unit 10 with respect to the subject eye E is confirmed, and a cross-sectional image of the subject eye front portion is captured in the alignment completion state. When the light source 102 of the cross-sectional imaging optical system 200 is turned on, the anterior segment of the subject eye is optically cut by the slit light from the slit 103. The return light from the optically cut anterior segment is imaged by the imaging element 107, so that a cross-sectional image of the anterior segment is acquired by the controller 2. The controller 2 analyzes the cross-sectional image by image processing, for example, acquires the positions of the anterior corneal surface and the posterior corneal surface of the corneal tissue. An analysis result may be output by being displayed on the display 15 together with the cross-sectional image.

Although a typical example of the present disclosure has been described above, the present disclosure is not limited to the above-described example, and various modifications can be made. For example, the first pattern index 23 is used as the ring index. The first pattern index 23 is not limited to the ring index, and for example, dot-shaped indexes may be arranged in a ring shape. The first pattern index 23 may be any shape and arrangement of a pattern index capable of measuring the corneal shape.

Further, in the example described above, the double ring index is formed on the second pattern index plate 72. The index of the second pattern index plate 72 is not limited to a double ring. The index of the second pattern index plate 72 may be, for example, an index provided with a plurality of points. The index of the second pattern index plate 72 may have a shape and an arrangement capable of measuring the corneal shape.

In the example described above, the illumination light source 51 is infrared light or white light. The illumination light source 51 may be, for example, a light source that emits one of infrared light and white light. The light source may emit both infrared light and white light.

In the example described above, the optical element of the optical paths coupler is one dichroic mirror. The optical element of the optical paths coupler may include a plurality of members.

In the example described above, the iris color information of the subject eye is acquired by performing image processing on the anterior segment captured image of the subject eye E captured by turning on the anterior segment illumination optical system 50. For example, the iris color information of the subject eye may be acquired by performing image processing on the captured image of the subject eye E onto which the index image of the pattern index is projected. The iris color information of the subject eye may be acquired before the weighting processing.

In the example described above, the controller 2 separates the color channel image. For example, the separation of the color channel image may be performed based on generation of a color signal by an image sensor or the like of the front imaging optical system 60. The front imaging optical system 60 may separate the color channel image by using an image sensor on which an image processing engine (not shown) is mounted. The image processing engine generates image data and performs various processes on the image data. The ophthalmic apparatus 1 may include an image processing engine separately from the controller 2. In this case, the image processing engine may separate the color channel image instead of the controller 2.

In the example described above, the imaging element 63 of the front imaging optical system 60 is a CCD image sensor. The imaging element 63 may be a complementary metal oxide semiconductor (CMOS) image sensor.

In the example described above, the illumination light source 32 is a white LED. The light of the illumination light source 32 may be visible light including light of a plurality of wavelengths. For example, red violet visible light obtained by mixing red light and blue light may be used.

Further, in the example described above, the iris color information of the subject eye E is acquired by the controller 2 performing image processing on the captured image. The controller 2 may acquire the iris color information by the examiner operating the operation unit 16 of the display 15 to input the iris color information of the subject eye. The controller 2 may acquire iris color information from an external device, for example. Further, the controller 2 may perform the image processing of the captured image without using the iris information. In this case, the controller 2 may perform image processing by calculating a weighting coefficient based on luminance information of each color channel of the captured image, for example.

In the example described above, the controller 2 acquires the weighting coefficient of each color channel image set for each iris color based on the iris color information. The controller 2 may calculate the weighting coefficient of each color channel image based on the luminance information (FIGS. 7A to 7C) of each color channel image. For example, the controller 2 may refer to the luminance information of the iris portion and the index image of the pattern index in each color channel image, and may use a larger weighting coefficient for a color channel image less affected by the iris color. Further, the controller 2 may use a weighting coefficient for each color channel image based on the contrast (luminance ratio) of each color channel image. For example, the controller 2 may use a larger weighting coefficient for a color channel image with high contrast. The controller 2 may acquire the weighting coefficient of each color channel image set based on the contrast level.

The controller 2 may use both the iris color information and the luminance information of each color channel image when acquiring the weighting coefficient. Alternatively, both the iris color information and the contrast of each color channel image may be used. By combining these information, the analysis processing can be efficiently performed. For example, the iris color information of the opposite eye of the subject eye E may be used as a reference using the iris color information once analyzed. The analysis processing of the opposite eye of the subject eye E may be omitted, and the iris color information of the subject eye E may be used for the analysis processing of the opposite eye. Further, for example, the iris color information of the subject eye E once analyzed may be called in the next and subsequent measurements of the subject eye E. Since the iris color analysis processing can be omitted by calling the iris color information, the efficiency of the analysis processing can be improved.

In the above-described example, the corneal shape information is acquired by performing image processing using a plurality of color channel images. For example, only one color channel image in which the weight value > 0 may be used to acquire the corneal shape information. In this case, the controller 2 may acquire the corneal shape information based on the color channel image itself.

In the example described above, the measurement result and the analysis result are output by being displayed on the display 15. For example, the output may be performed by printing on a printing sheet using a printer. In addition, for example, data may be output by transferring the data to a computer in which a database is stored via data communication means.

What is claimed is:

1. An ophthalmic apparatus, comprising:
an examination window; a first pattern index projection optical system configured to project a first pattern index onto a subject eye from an outside of the examination window; a second pattern index projection optical system configured to project a second pattern index onto the subject eye from an inside of the examination window; an imaging optical system including an imaging element configured to receive a first cornea reflection image caused by the first pattern index and a second cornea reflection image caused by the second pattern index via an objective lens; and
an optical paths coupler configured to couple optical paths of the imaging optical system and the second pattern index projection optical system, wherein
the first index projection optical system projects the first pattern index with visible light, the second index projection optical system projects the second pattern index with infrared light, and the optical paths coupler is an optical element having a characteristic in which a ratio of attenuation of the visible light is smaller than a ratio of attenuation of the infrared light in an optical path from a cornea of the subject eye toward the imaging element.

2. The ophthalmic apparatus according to claim 1, wherein the second pattern index is a ring-shaped pattern.

3. The ophthalmic apparatus according to claim 1, further comprising:
an anterior segment illumination optical system configured to illuminate an anterior segment of the subject eye, wherein a wavelength of a light source of the second pattern index projection optical system is identical to a wavelength of a light source of the anterior segment illumination optical system.

4. The ophthalmic apparatus according to claim 1, further comprising:
an anterior segment illumination optical system configured to illuminate an anterior segment of the subject eye, wherein a light source of the anterior segment illumination optical system is turned off while the second pattern index projection optical system projects the second pattern index.

5. The ophthalmic apparatus according to claim 2, further comprising:
an anterior segment illumination optical system configured to illuminate an anterior segment of the subject eye, wherein a wavelength of a light source of the second pattern index projection optical system is identical to a wavelength of a light source of the anterior segment illumination optical system.

6. The ophthalmic apparatus according to claim 4, wherein the second pattern index is a ring-shaped pattern.

7. The ophthalmic apparatus according to claim 4, wherein a wavelength of a light source of the second pattern index projection optical system is identical to a wavelength of a light source of the anterior segment illumination optical system.

8. The ophthalmic apparatus according to claim 4, wherein the second pattern index is a ring-shaped pattern, and a wavelength of a light source of the second pattern index projection optical system is identical to a wavelength of a light source of the anterior segment illumination optical system.
